(19) United States
(12) Patent Application Publication
(10) Pub. No.: US 2022/0280068 A1
(43) Pub. Date: Sep. 8, 2022

(54) SPIROMETRY SYSTEM FOR MITIGATING PHYSICAL USER ERRORS

(71) Applicant: Loop Medical Innovations Corp., Sugar Land, TX (US)

(72) Inventors: Ronit Kar, Lexington, KY (US); Ajay Sanjay Thatte, Flower Mound, TX (US); Eshan Mukesh Sayani, Highland Village, TX (US); Juan Enrique Villacres Perez, Spring, TX (US); Abhishek Dasgupta, Sugar Land, TX (US)

(21) Appl. No.: 17/653,590

(22) Filed: Mar. 4, 2022

Related U.S. Application Data
(60) Provisional application No. 63/156,705, filed on Mar. 4, 2021.

(51) Int. Cl. A61B 5/093; A61B 5/087

(52) U.S. Cl. CPC A61B 5/0935; A61B 5/087; A61B 2560/0271; A61B 2560/0462; A61B 2562/164; A61B 2560/028; A61B 2560/0487

(57) ABSTRACT

A spirometry system operable to measure airflow through an air passage defined by a mouthpiece including one or more contact sensors configured to generate at least one mouth contact signal indicative of user mouth contact with the proximal portion of the mouthpiece. The spirometry system can further include an airflow sensor configured to generate an airflow signal corresponding to airflow within the air passage, and processor circuitry communicatively coupled to the one or more contact sensors and to the airflow sensor. The processor circuitry can be configured to determine an indication of an amount of user mouth contact for use with a measurement of airflow by the processor circuitry.

### CLAIM OF PRIORITY

This application claims the benefit of U.S. Provisional Patent Application Ser. No. 63/156705, filed on Mar. 4, 2020, the benefit of priority of which is claimed hereby, and which is incorporated by reference herein in its entirety.

### BACKGROUND

Spirometry is a pulmonary function test for the diagnosis of many restrictive or obstructive respiratory diseases, such as asthma and COPD. Spirometry involves the patient taking a deep breath and exhaling as intensely as possible for as much time as they can into the mouthpiece of the spirometer, which collects data on patient respiration by evaluating differences in pressure within the spirometer to calculate mass air flow. The volume of expiration (L) and flow rate (L/s), as well as the time (s) and volume (L) are illustrated on spirograms that display the data of the expiration and the following inspiration. Some of the notable aspects of this data for the diagnosis of obstructive lung diseases are the forced vital capacity (FVC) and the forced expiratory volume (FEV1).

### BRIEF DESCRIPTION OF THE DRAWINGS

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1 is a front view of a system for conducting spirometry according to some embodiments.

FIG. 2 is a perspective, side view of a handheld spirometer with additional sensors according to some embodiments.

FIG. 3 is an enlarged perspective view of a portion of the mouthpiece of the device of FIG. 2 according to some embodiments.

FIG. 4 is a side view of a portion of the device of FIG. 2 wherein the part is shown as a perspective, transparent wireframe to depict an internal airflow chamber along with the functional sensor array.

FIG. 5 is a diagram of the electrical element of the device of FIG. 2 and a receiver of the system in FIG. 1 according to some embodiments.

FIG. 6 is a perspective view of a handheld spirometer with additional mechanical mechanisms according to some embodiments.

FIG. 7A illustrates a perspective view of an example of a spirometry system.

FIG. 7B illustrates a perspective view of an example of a mouthpiece of a spirometry system.

FIG. 8 illustrates a block diagram of an example of a spirometry system communicatively coupled to an external device.

FIG. 9 illustrates a process for mitigating physical user-errors during use of a spirometry system.

FIG. 10 illustrates a flow chart of an example of a method for mitigating physical user-errors during use of a spirometry system.

### SUMMARY/OVERVIEW

To perform pulmonary function testing, spirometer systems are used by blowing into a mouthpiece that includes or communicates with an airflow sensor. The airflow sensor communicates with a computational element (e.g., processor circuitry) to measure a pressure differential, such as between locations before and after a flow restriction in the mouthpiece, to determine air flow. It is helpful to increase the portability and connectivity of spirometer systems. For example, personal spirometers, relative to their conventional clinical or point-of-care counterparts, can include portable handheld devices including a mouthpiece that a user blows into, with supporting electronic components to enable airflow data collection and analysis. Personal spirometers can be connectible to a mobile phone, tablet, or laptop computer such as to allow for the output and display of the resulting measurements to a user.

However, certain approaches to both conventional point-of-care and personal spirometry systems can face many problems that can lead to inaccurate measurements of various values or metrics essential to the diagnosis of obstructive and restrictive lung diseases. For example, some patients push their tongue against the mouthpiece while exhaling, which increases the initial pressure for airflow through the mouthpiece and can lead to an inaccurately high measured FEV1/FVC ratio. Conversely, if a patient does not position their mouth on the mouthpiece in manner that forms a complete lip seal, there can be a leakage of air that generates an inaccurately low measured FEV1/FVC ratio. These physical user problems, along with many others, can significantly impede an accurate diagnosis of an obstructive condition in a patient by a physician. Further, in the absence of a physician or technician coaching a patient on the proper use of the mouthpiece, the likelihood of such physical user problems impeding proper diagnosis of respiratory conditions increases, such when a patient uses a personal spirometer at home.

As such, clinical or point-of-care spirometry tests with a trained physician or technician are desirable to prevent physical user errors that can result in a misdiagnosis. However, clinical or point-of-care appointments are often expensive and are less convenient for a patient. Further, such appointments can be difficult for medical professionals to schedule and perform in-person tests within a desirable time period. For example, clinical access can be limited by high demand, government restrictions, such as during a pandemic or public health crisis, or other circumstances. This can exacerbate the already significant hurdles both patients and healthcare providers face in obtaining or providing ongoing respiratory health monitoring or post-operative follow up assessments. Accordingly, an error-minimizing spirometer that can detect various sources of physical user error (e.g., improper lip sealing around device mouthpiece, cough during test procedure, improper tongue placement or motion on device mouthpiece, etc.) to thereby eliminate the need for physician coaching a user during in-person lung function testing appointment is desirable.

The present disclosure can address these issues, among others, by providing a spirometry system including one or more contact sensors on or near the mouthpiece to detect and correct for user physical errors. For example, the mouthpiece can include a plurality of contact sensors distributed radially (or otherwise peripherally) around an outer surface of the mouthpiece such as to sense an amount of user lip contact with the mouthpiece and can include a force sensor to sense an amount of user tongue contact with the mouthpiece. The spirometry system can further recognize additional user or system errors such as by comparing or otherwise using at least one signal from the one or more contact sensors with at least one signal from an airflow sensor, such as to detect incorrect calibration, such as positive or negative calibration before a test, a late start to a test, a cough during a test, sub-maximal inhalation during a test, sub-maximal blast during a test, early user termination during a test, variable effort during a test, cessation of airflow during a test, tongue thrusting during a test, among others.

The spirometry system can further be in communication with an external device such as a mobile phone or tablet running a proprietary or other mobile application to cause the external device to notify the user, such as via an audible or visual alert, or haptic alert, if any type of user or system error is detected. The spirometry system of the present disclosure can thereby help to address certain common physical user and system errors associated with point-of-care and portable spirometry systems to help users more easily and consistently generate and collect high-quality, error-free data associated with pulmonary function.

### DETAILED DESCRIPTION

The following description and the drawings sufficiently illustrate specific examples to enable those skilled in the art to practice them. Other examples can incorporate structural, process, or other changes. Portions and features of some examples can be included in, or substituted for, those of other examples. Examples set forth in the claims encompass all available equivalents of those claims.

The present disclosure has recognized that current spirometer devices include the existence of various sources of user error (e.g., improper lip sealing around device mouthpiece, cough during test procedure, improper tongue placement or motion on device mouthpiece, etc.). The present disclosure has further recognized that existing devices are unable to detect or correct such problems. To mitigate, through detection or prevention, the device outlined herein aims to help inhibit or prevent or to detect or quantify sources of error in such respiratory testing based on spirometry or other relevant testing protocols using various possible arrangements of sensors on or near the mouthpiece of such a device.

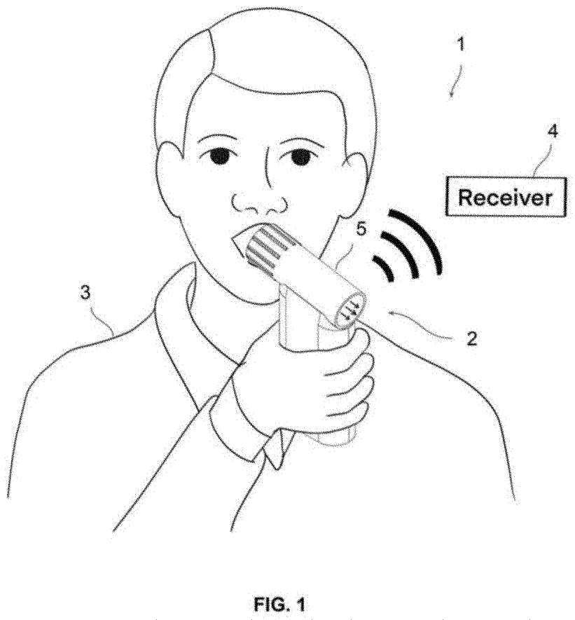

Referring to FIG. 1, illustrated is an embodiment of a system 1 configured, manufactured and operated according to one or more aspects of this disclosure. The system 1 of FIG. 1 includes a device 2 that can be utilized by user 3 and has the capability to transmit data to a receiver 4 of a peripheral device. Operation may involve he user 3 placing their mouth on the mouthpiece 5 and blowing air such as for lung function testing.

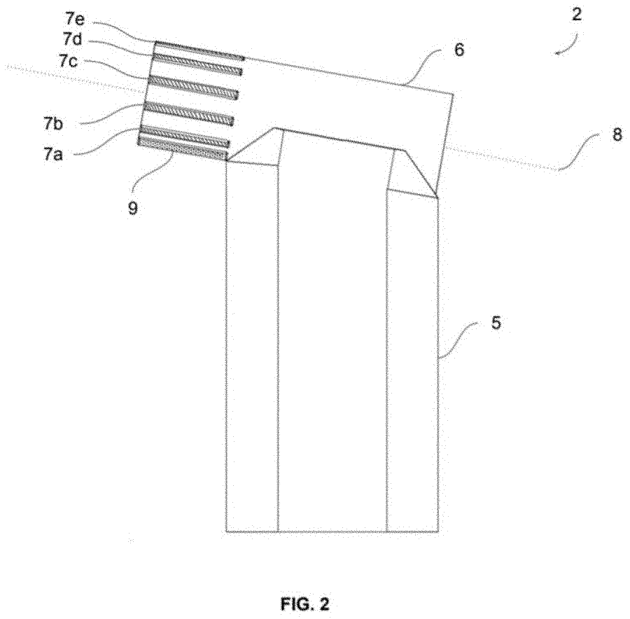

Referring now to the embodiment of FIG. 2, the device 2 includes a body 5 and an attached mouthpiece 6. The mouthpiece 6, in the illustrated embodiment, has lip sensors 7a, 7b, 7c, 7d, 7e running radially along the central axis of the mouthpiece 8 as well as tongue sensor 9 externally placed on or distributed about the mouthpiece 6. From here on, all lip sensors may be referred collectively as 7.

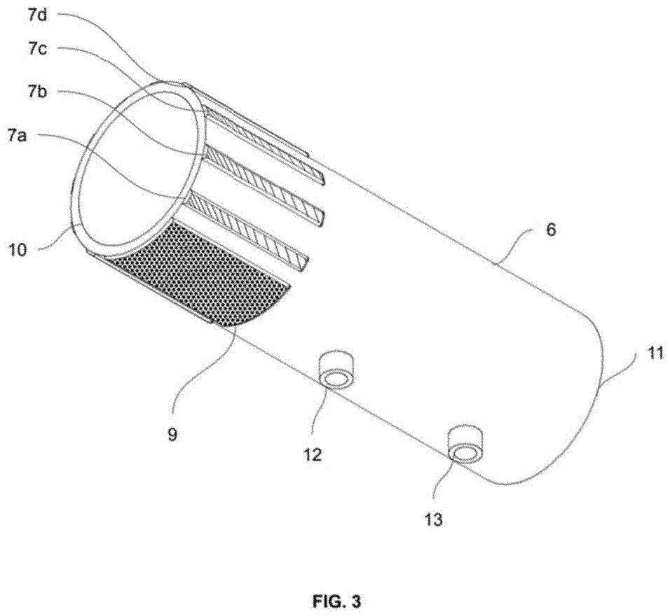

Referring now to the embodiment of FIG. 3, mouthpiece 6 may embody a hollow tube that includes a proximal mouth placement site 10 and a distal air expulsion site 11. The mouthpiece 6 may house a sensor for measuring air flow through the chamber that may utilize a number of outlets 12 and 13. These ports 12 and 13 may involve connection to the device body 5. The mouthpiece 6 may include lip sensors 7 that are radially placed on the exterior of the proximal mouth placement site 10. The mouthpiece 6 may also include a tongue sensor 9 placed on the exterior of the proximal mouth placement site.

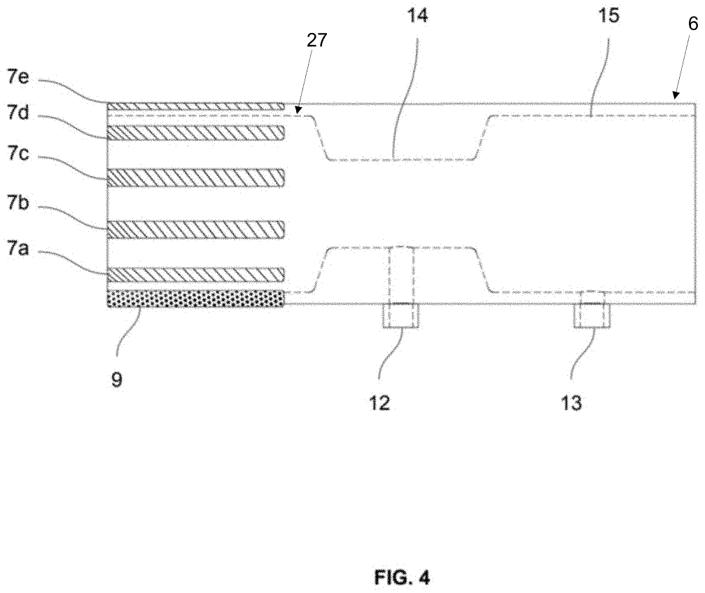

Referring now to the embodiment of FIG. 4, air flow measurement may be conducted by utilizing air flow dynamics in multiple chambers (e.g., multiple different sized chambers). The mouthpiece 6 may include or be coupled to internal chambers 14 and 15 that allow for measurement of air flow through ports 12 and 13 respectively. Sensors on the proximal end of the mouthpiece 5 including lip sensors 7 and tongue sensor 9 may transmit data using electrically-conductive channels that may run through the wall of the mouthpiece 6. These channels may terminate at pads used for data transmission with the electronics system housed in the device body 5.

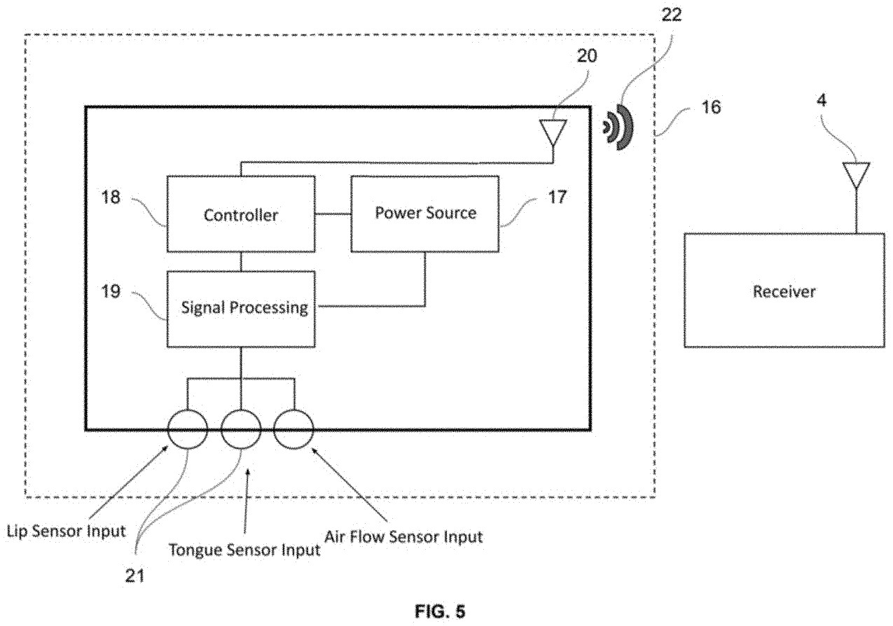

Referring now to the embodiment of FIG. 5, electronics system 16 may consist of a power source 17, an appropriate controller 18, a signal processing system 19, and an antenna 20. Electronics system 16 may also include ports 21, where sensors 7, 9, and an airflow sensor can connect. Data may be relayed through the electronics system 16 to an external receiver 4 using wired connections or through wireless communication 22.

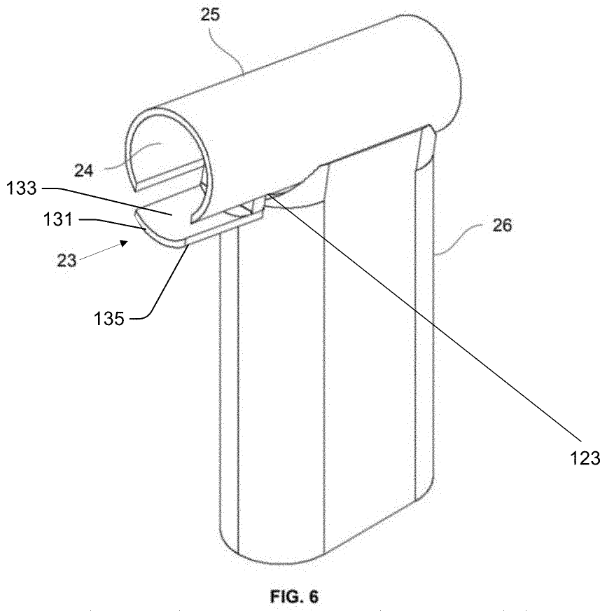

Referring now to the embodiment of FIG. 6, an alternative aspect of device 2 may include a mechanical component 23 to detect tongue placement or other sources of user error instead of digital sensors 7 and 9 as demonstrated by the embodiment in FIG. 3. In this embodiment, the mechanical component 23 completes the air chamber 24 housed in a mouthpiece 25 housed on the device body 26. The completion of this air chamber 24, in one or more embodiments, can help allow for unobstructed air flow used for lung function evaluation.

Airflow in device 2 may be detected via internal sensors in or in fluid communication with mouthpiece 6, which may incorporate differential pressure sensors, ultrasound sensors, or any other appropriate sensor (e.g., digital or otherwise), as shown in the embodiment of mouthpiece 6 in FIG. 3. These digital sensors may monitor airflow through entry port 12 and 13 and may do so without disrupting the air flow in any significant manner.

Furthermore, the sensors may interface with the device body 5 through ports 12 and 13 as depicted in the embodiment of FIG. 4 or through any other suitable configuration. In alternative embodiments of device 2, there may be mechanical systems and sensors for detecting airflow instead of digital sensors, as highlighted in FIG. 6.

The sensor array, which may include sensors 7 and 9, may detect one or both of mouth and tongue placement at the proximal mouth placement site 10 on the mouthpiece 6. One purpose of the sensor array is to help ensure that no tongue obstruction and optimal mouth sealing occurs at the proximal mouth placement site 10 during lung function testing. The signals detected by sensors 7 and 9 may be transmitted to the electronics housed in the device body 5 through internal channels.

The device 2 may be controlled by an electronics system, which performs one, more, or all of the desired computations. These may include collecting airflow data, determining the volume of exhalation, the speed of the exhalation, and other parameters important in respiratory health. The electronics system may also evaluate various errors during use. Furthermore, information may be relayed between the device 2 and mobile devices capable of supporting applications, including but not limited to smart phones and tablets. This information can also be transferred to physicians, such as via HIPAA compliant programs.

The tongue sensor 9 and lip sensors 7, in one or more embodiments, relay contact information directly to the central electronics system for evaluation. The airflow sensor is coupled (e.g., directly coupled in one or more embodiments) to the central electronics system and relays the airflow data that may be obtained through ports 12 and 13 on mouthpiece 6 to the electronics system wherein calculations may be performed and data processed for transfer through the antenna 20 to a receiver 4 or transfer via direct connection to an interface. The receiver 4 may be coupled to an external device for data transfer. In some embodiments, a mechanical mechanism 23 may be used to detect the placement of the tongue of the device user 3. For instance, reference is now made to FIG. 6 where in the mechanical mechanism 23 is used in mouthpiece 25 in the system 1 in place of sensor 9. The mechanism may operate by motion (e.g., upwards motion in one embodiment) of the attached component 23 triggering the completion of airway 24 within mouthpiece 25. By doing so, functional operation of the device 2 may be enabled so as to only be possible once the user 3 pushes on this component 23 with their tongue.

In some embodiments, a mechanical mechanism 23 may be used to detect the placement of the tongue of the device user 3. For instance, reference is now made to FIG. 6 where in the mechanical mechanism 23 is used in mouthpiece 25 in the system 1 in place of sensor 9. The mechanism may operate by motion (e.g., upwards motion in one embodiment) of the attached component 23 triggering the opening of airway 24 within mouthpiece 25. By doing so, functional operation of the device 2 may be enabled so as to only be possible once the user 3 pushes on this component 23 with their tongue.

As per the above disclosure, the embodiments described herein include a tool for pulmonary function testing that may comprise an airflow chamber that may house sensors or other airflow detection mechanisms, an array of sensors or other airflow or error detection mechanism with various possible arrangements, an electronics system that may collect relevant information, and connections relevant to the system that may be arranged in various forms. The embodiment herein may allow for pulmonary function testing and mitigation of various sources of user error through detection or prevention as previously described.

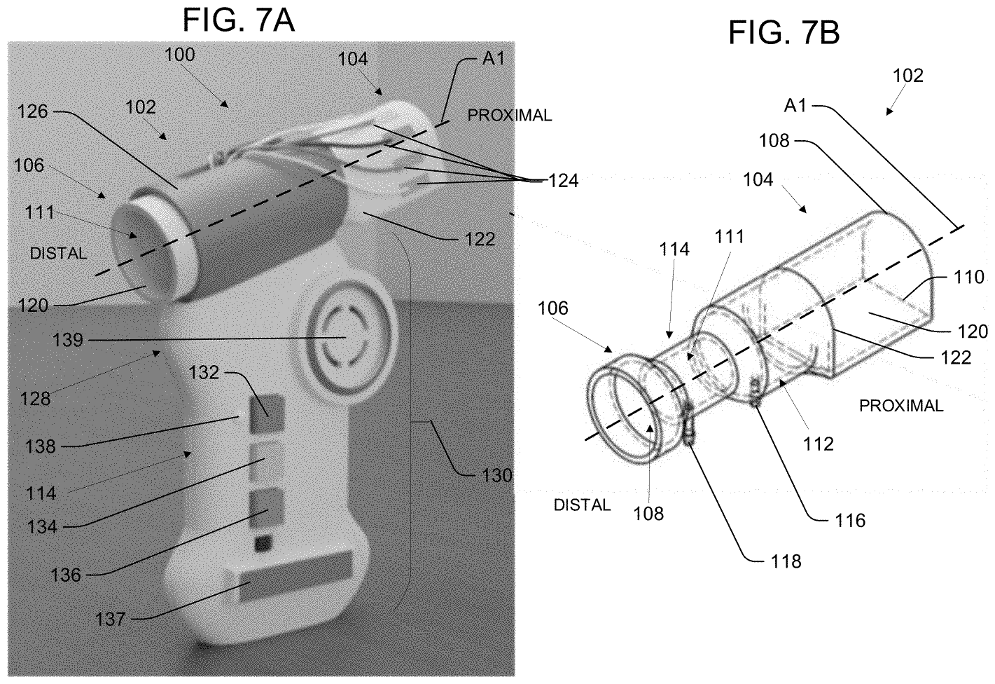

FIG. 7A illustrates a perspective view of an example of a spirometry system 100. FIG. 7B illustrates a perspective view of an example of a mouthpiece 102 of a spirometry system 100. In FIG. 7B, the mouthpiece 102 is shown in shadow. FIGS. 7A-7B include a dashed line corresponding to a central axis A1 defined by the mouthpiece 102, and orientation indicators “Proximal” and “Distal”. FIGS. 7A-7B are discussed below concurrently. The spirometry system 100 can include, or can otherwise be similar to, any of the systems, devices, or components shown in and discussed with regard to FIGS. 1-6 above. The spirometry system 100 can include a mouthpiece 102. The mouthpiece 102 can be similar to the mouthpiece 6 or to the mouthpiece 25 shown in and described with regard to FIGS. 2-4 or FIG. 6 above.

The mouthpiece 102 can define the central axis A1. The mouthpiece 102 can be a reuseable or a disposable (e.g., a single use) mouthpiece. The mouthpiece 102 can be generally tubular or cylindrical in shape, but can also comprise various other three-dimensional shapes such as an ellipsoidal prism, a rectangular prism, or any combination thereof. The mouthpiece 102 can include a proximal portion 104 and a distal portion 106. In an example, the proximal portion 104 can form a doghouse shape, and the distal portion 106 can form a tubular or cylindrical shape. In such an example, the proximal portion 104 can define a curved surface 108 (FIG. 7B) forming a semi-circular shape and a linear surface 110 (FIG. 7B) forming a flat or flattened shape, such as extending along an axis parallel to and laterally offset from the central axis A1. The curved surface 108 can comprise about, but is not limited to, 70-80, 81-90, or 91-100 degrees of a total circumference of the proximal portion 104 of the mouthpiece 102.

The mouthpiece 102 can define an air passage 111 extending through the mouthpiece 102 between the proximal portion 104 and the distal portion 106. The air passage 111 of the mouthpiece 102 can include at least a first chamber 112 (FIG. 7B) and a second chamber 114 (FIG. 7B) in fluid communication therewith. The air passage 111 can be similar to the internal airflow chamber 27 shown in FIG. 4, for example, the first chamber 112 can be concentrically defined with the second chamber 114 by the air passage 111 of the mouthpiece 102. The first chamber 112 and the second chamber 114 can generally be tubular or cylindrical in shape, and the first chamber 112 can define a larger, or increased, diameter relative to the second chamber 114, such that air flow measurement can be conducted by utilizing or otherwise analyzing a difference in air flow dynamics between two different sized chambers.

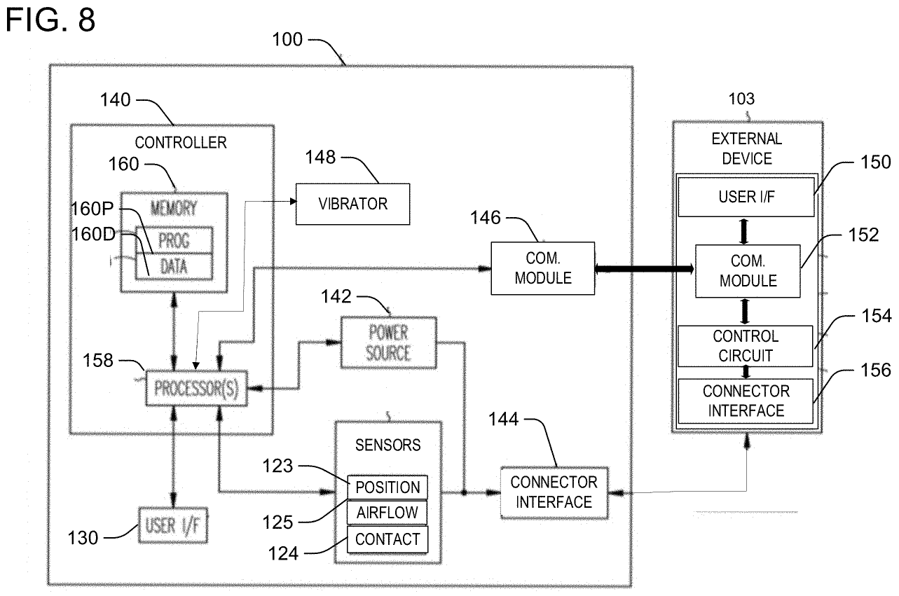

For example, the first chamber 112 can include a first port 116 (FIG. 7B) and the second chamber 114 can include a second port 118 (FIG. 7B). The first port 116 and the second port 118 can be similar to the ports 12 and 13 shown in FIGS. 3-4, at least in that the first port 116 is configured to allow airflow out of the first chamber 112 for measurement and the second port 118 is configured to allow airflow out of the second chamber 114 for measurement, such to allow the airflow sensor 125 shown in FIG. 8 below to be in fluid communication with the air passage 111. For example, the first port 116 and the second port 118 can be tubular passages extending through the mouthpiece 102 between an inner surface 120 and an outer surface 122 thereof. In some examples, at least a portion of the first port 116 and the second port 118 can be configured to be received within a tube, such as to help reduce the possibility of inaccurate measurement due to an air leak between the first port 116 or the second port 118, and the airflow sensor 123 (FIG. 8).

The mouthpiece 102 can include one or more contact sensors 124 (FIG. 7A & 8). The one or more contact sensors 124 can be similar to the lip sensors 7A-7D or the tongue sensor 9 shown in FIG. 3, at least in that the one or more contact sensors 124 can be configured to sense physical user contact with the mouthpiece 102 and generate at least one mouth contact signal indicative of user mouth contact with the proximal portion 104 of the mouthpiece 102. For example, the one or more contact sensors 124 can include any of, but not limited to, a capacitive contact sensor, a resistive contact sensor, an optical proximity sensor, a resistive force sensor, or a load cell. The one or more contact sensors 124 can include any suitable number, such as, but not limited to, one, two, three, four, five, six, seven, eight, nine or ten contact sensors. The one or more contact sensors 124 can be fixedly coupled to the proximal portion 104 of the mouthpiece 102, such as by being embedded in or molded into the mouthpiece, or by an adhesive or other fixation means.

The one or more contact sensors 124 can protrude laterally beyond the outer surface 122 of the mouthpiece 102 or can be recessed into or otherwise embedded into the mouthpiece 102, such that the one or more contact sensors 124 are located at least partially within the mouthpiece 102. For example, an outward-most surface (e.g., a surface of each of the one or more contact sensors 124 facing away from the mouthpiece 102) can extend or otherwise be located flush with or sub-flush with the outer surface 122.

The one or more contact sensors 124 can be distributed about the mouthpiece 102 in various arrangements. For example, the one or more contact sensors 124 can be distributed about the proximal portion 104 of the mouthpiece in an annular, radial, or otherwise circular arrangement. In such an illustrative example, relative to the central axis A1, each of the one or more contact sensors 124 can be spaced apart from each other (e.g., at equal or different intervals) such as by between about 1 and 180 degrees or 36 and 180 degrees, depending on the number of individual sensors the one or more contact sensors 124 includes. For example, if the one or more contact sensors 124 includes four individual sensors, then a surface of a first contact sensor of the one or more contact sensors can be located at about 90 degrees relative to a corresponding adjacent surface of a second and adjacently located contact sensor 124 of the or more contact sensors 124. If the one or more contact sensors 124 includes eight individual sensors, then a surface of a first contact sensor of the one or more contact sensors can be located at about 45 degrees relative to a corresponding adjacent surface of a second and adjacently located contact sensor 124 of the or more contact sensors 124.

In some examples, the one or more contact sensors 124 can be distributed about the proximal portion 104 of the mouthpiece in a semi-circular arrangement. For example, at least one of the one or more contact sensors 124 can be located on, or otherwise located with respect to, the linear surface 110, such as shown in FIG. 3, which in an example can comprise about 90 degrees of a total circumference of the proximal portion 104. In such an example, relative to the central axis A1, each of the one or more contact sensors 124 can be spaced apart from each other by between about 27 and 135 degrees, depending on the number of individual sensors the one or more contact sensors 124 includes. For example, if the one or more contact sensors 124 includes four individual sensors, then a surface of a first contact sensor of the one or more contact sensors can be located at about 67.5 degrees relative to a corresponding adjacent surface of a second contact sensor 124 of the or more contact sensors 124. If the one or more contact sensors 124 includes eight individual sensors, then a surface of a first contact sensor of the one or more contact sensors can be located at about 33.75 degrees relative to a corresponding adjacent surface of a second contact sensor 124 of the or more contact sensors 124. In still further examples, the one or more contact sensors 124 can be located at non-equidistant spacings, relative to one another, around the central axis A1, such as, but not limited to, to bias sensitivity toward upper lip contact or seal, lower lip contact or seal, continuous tongue contact or movement, or a combination thereof.

The one or more contact sensors 124 can also be selectively arranged on the proximal portion to help encourage ergonomic contact with a user's lips or mouth. In an example, the one or more contact sensors 124 located on the curved surface 108 can include at least one capacitive contact sensor or an optical proximity sensor to indicate an amount of contact with a patient's lip or lips along the curved surface 108, such by how many of the one or more contact sensors 124 indicate lip contact therewith, and the one or more contact sensors 124 located on the linear surface 110 can be a resistive force sensor or a load cell to measure an amount of pressure a user's tongue exerts on the linear surface 110 of the mouthpiece 102, such as to generate a tongue contact signal to provide an indication of an amount of user tongue contact with the proximal portion 104.

The one or more contact sensors 124 can be communicatively coupled to various electronic components of the spirometry system 100, such as to the controller 140 circuitry shown in and described with regard to FIG. 8. For example, each of the one or more contact sensors 124 can include a wire extending between each of the one or more contact sensors and an input on a controller, such the ports 21 of the controller 18 shown in FIG. 5, or other electronic components, such as a separate intermediary controller located between the one or more contact sensors 124 and the processor circuitry of the controller 140 (FIG. 8) configured to process or otherwise input or output signals from the one or more contact sensors 124.

In some examples, such as shown in and discussed with regard to FIG. 6 above, the proximal portion 104 of the mouthpiece 102 (e.g., mouthpiece 25 in FIG. 6) can include a mechanical mechanism 23 (FIG. 6) and a position sensor 127 (FIG. 6). The mechanical mechanism 23 can generally be body translatable or otherwise movable relative to the central axis A1 (FIG. 7A) defined by the air passage 111 (FIG. 7A). The mechanical mechanism can define member 131. The member 131 can be a portion of the mechanical mechanism 23 configured to contact the tongue of a user, and can extend along an axis parallel to, and laterally offset from, the central axis A1.

The mechanical mechanism 23 can include one or more components or features to enable the member 131 to be translated or otherwise moved between a first position and a second position. In an example, the member 131 can translate or slide along an axis extending orthogonally to the central axis A1 between the first position and the second position. In the first position, as shown in FIG. 6, the member of the mechanical mechanism 23 can be spaced laterally apart from the central axis A1 (FIG. 7A) defined by the air passage 111 (FIG. 7A). In the second position, the member 131 can form a portion of the outer surface 122 (FIG. 7A) of the mouthpiece 102 when the member 131 is in the second position. For example, an inner surface 133 or an outer surface 135 of the member 131 can align or become flush with the inner surface 120, or the outer surface 122, of the mouthpiece when the member 131 is in the second position. In some examples, the outer surface 135 of the member 131 can include at least one or the one or more contact sensor 124.

The position sensor 123 can include one or more sensors configured to generate at least one member position signal indicative of a position of the member 131. The position sensor 127 can be, but is not limited to, a capacitive contact sensor, such as operable to detect contact between a first component located on the mechanical mechanism 23 (FIG. 6) and a second component located on or within the mouthpiece 102 or a housing 128 (FIG. 7A), a resistive contact sensor, an optical proximity sensor, an encoder, a magnetostrictive sensor, or others. The position sensor 127 can be in communicative contact with other electronic components of the spirometry system 100, such with controller 140 shown in and described with regard to FIG. 8.

The spirometry system 100 can include a sleeve 126 (FIG. 7A). In FIG. 7B, the mouthpiece 102 is shown with the sleeve removed. The sleeve 126 can circumferentially encompass at least a portion of the mouthpiece 102, such as generally between the proximal portion 104 and the distal portion 106 in a position distal to the one or more contact sensors 124. The spirometry system 100 can include a housing 128. The housing 128 can be similar to the body 5 shown in, and described with respect to, FIGS. 1-2 above. The housing 128 can form a handle-like shape, but can form a variety of other three-dimensional shapes, such as ellipsoidal or rectangular prisms.

The housing 128 can be configured to be fixedly or removably coupled to the mouthpiece 102, such to the first port 116, the second port 118, the outer surface 122, the sleeve 126, or to others portion of the mouthpiece 102 via various end-user-attachment or end-user-detachment elements such as, but not limited to, magnetic attachment, one or more latches, a snap fit, or others. In some examples, the housing 128 can be configured to receive a portion of the mouthpiece 102 within the sleeve 126, such as to help couple the mouthpiece 102 to the housing 128.

The housing 128 and the mouthpiece 102 can each include any of wiring, one or more quick connectors, such as one or more pins, one or more magnetic connections, one or more latched connections, one or more soldered connections, one or more electrical contacts (such as brass or copper), conductive tape, or one or more other electrical connection elements to enable the one or more contact sensors 124 to establish electrical communication with various electrical components of the housing 128, such as the controller 140 shown in and described with regard to FIG. 8 below.

The housing 128 can include a user interface 130. The user interface 130 can be configured to initiate or trigger various operations of the spirometry system 100. In an example, such as shown in FIG. 7A, the user interface 130 can include a first button 132, a second button 134, a third button 136, a slide switch 137, a light emitter 138, and an audio driver 139. The light emitter 138 can be for example, not limited to, a light emitted diode (LED). In certain examples, the user interface 130 can include more than one light emitter 138, an increased or decreased number of buttons or switches, or other user input or output features, such as a display screen or touchscreen.

In the operation of some examples of a spirometry system 100 including the user interface 130 shown in FIG. 7A, a user can start a test procedure by depressing the first button 132 to initiate communication between the spirometry system 100 and an external device, such as the external device 103 shown in and described with regard to FIG. 8 below. Until wireless communication between the spirometry system 100 and an external device is established, the light emitter 138 can flash to indicate that the spirometry system 100 is not yet paired with the external device. Once wireless communication is established between the spirometry system 100 and the external device 103, the light emitter 138 can be continuously illuminated. A user can then depress the second button 134 to initiate a timed countdown before the test procedure begins. A user can subsequently follow additional prompts or view the results of the test using the external device 103, such by utilizing a proprietary mobile application running on the external device 103. A user can optionally depress the third button 136 to end a test procedure part-way through or can move the slide switch 137 to control the volume level of the audio driver 139. In other examples, the spirometry system 100 can be configured to perform the test procedure or any operations discussion in this disclosure as a standalone unit, such as without communication with the external device 103.

The mouthpiece 102, the housing 128, or various other components of the spirometry system 100, can include or be made from, in an example, one or more plastics or one or more composites. For example, the components listed above can include or be made from PLA or ABS plastic. The mouthpiece 102, the housing 128, or various other components of the spirometry system 100 can be made from stainless steel or other metals, such as via machining or metallic molding. Further, the mouthpiece 102, the housing 128, or various other components of the spirometry system 100 can be manufactured using any compatible fabrication technique such as die-casting, injection molding, lithography, SLA printing, 3D printing, or others.

FIG. 8 illustrates a block diagram of an example of a spirometry system 100 communicatively coupled to an external device 103. FIG. 8 is discussed with reference to the spirometry system 100 shown in, and described above with regard to, FIGS. 7A-7B. In some examples, the spirometry system 100 can be realized using the elements shown in FIG. 8. In other examples, the spirometry system 100 can include other elements. As illustrated in FIG. 8, the spirometry system 100 can include any of a position sensor 123, one or more contact sensors 124, an airflow sensor 125, a user interface 130, a controller 140, a power source 142, a connector interface 144, a communication module 146, a piezoelectric or other vibrator 148, and the external device 103 can include a user interface 150, a communication module 152, a control circuit 154, and a connector interface 156. The external device 103 can be for example, but not limited to, a mobile phone, a tablet, a laptop or desktop computer.

The controller 140 can include at least a processor 158 and a memory 160. In some examples, the processor 158 can include a timer and/or a clock. The processor 158 can include a hardware processor, such as any of central processing unit (CPU), a graphics processing unit (GPU), a hardware processor core, or any combination thereof.

The processor 158 can include any one or more of a microprocessor, a control circuit, a digital signal processor (DSP), an application specific integrated circuit (ASIC), a field-programmable gate array (FPGA), or other equivalent discrete or integrated logic circuitry. The processor 158 can thereby be capable of receiving, retrieving, and/or processing program instructions, such as stored on the memory 160 (e.g., on program memory 160P), or receiving, retrieving, and/or processing data stored on the memory 160 (e.g., on data memory 152D), or on data memory of the control circuit 154 of the external device 103 to implement or otherwise execute any of, but not limited to, the various functions or operations of the spirometry system 100 described in the present disclosure.

In some examples, the memory 160 can be described as volatile memory, meaning that the memory 160 does not maintain stored contents when power to the spirometry system 100 is turned off. Examples of volatile memories can include random access memories (RAM), dynamic random-access memories (DRAM), static random-access memories (SRAM), and other forms of volatile memories. In some examples, the memory 160 can include one or more computer-readable storage media. In some examples, the memory 160 can be configured to store larger amounts of information than volatile memory. In some examples, the memory 160 can further be configured for long-term storage of information. In some examples, the memory 160 can include non-volatile storage elements. Examples of such non-volatile storage elements can include magnetic hard discs, optical discs, flash memories, or forms of electrically programmable memories (EPROM) or electrically erasable and programmable (EEPROM) memories.

The user interface 130 or the user interface 150 can communicate or transfer information between the spirometry system 100 and a user (e.g., a patient or technician). For example, the user interface 130 or the user interface 150 can include input and output devices such as a visual display, audible signal generators or audio drivers, switches, buttons, or a touchscreen. The communication module 146 can enable wireless (e.g., network) communication with the external device 103, such as to transmit or receive data via near-field communication (NFC), Bluetooth (e.g., Bluetooth Low Energy), Wi-Fi, 3GPP LTE, or any other wireless communication protocol, which can optionally include one or more healthcare compliant, such as HIPPA compliant, aspects. In some examples, the communication module 146 can also include a network interface card, a wireless communication interface, an optical transceiver, a radio frequency transceiver, or any other type of device that can send and receive information. As such, the user interface 130 can utilize the communication module 146 to, for example, send information to or receive information from the communication module 152 of the external device 103. The connector interface 144 can allow for a direct or wired connection with the external device 103. For example, the connector interface 144 can be configured to enable direct communication between the spirometry system 100 and the external device 103 via cable. In some examples, the connector interface 144 can also allow for the input for power to the power source 142 from the external device 103, or from other types of devices such as a transformer.

The position sensor 123 and the one or more contact sensors 124 can be similar to the examples described with regard to FIGS. 7A-7B above. The airflow sensor 125 can be a differential pressure sensor. For example, the airflow sensor 125 can be, but is not limited to, a differential pressure sensor positioned within the housing 128 and in fluid communication with the first port 116 (FIG. 7B) and the second port 118 (FIG. 7B). The power source 142 can provide electrical energy to any of the electrical components of the spirometry system 100. In an example, the power source 142 can be a battery located within the housing 128 (FIG. 7A). In other examples, the power source 142 can be a type of power source configured to provide power to the spirometry system 100 by receiving power from an external device, such as through the connector interface 144.

The vibrator 148 can be configured to provide a user with a tactile or haptic alert, such as by causing the housing 128 to vibrate. For example, the vibrator 148 can be, but is not limited to, an electric motor, a piezoelectric vibrator, or others. In some examples, the spirometry system 100 can further include, or can alternatively include in place of the vibrator, other devices configured to provide other types of tactile or haptic alerts, such as thermal feedback.

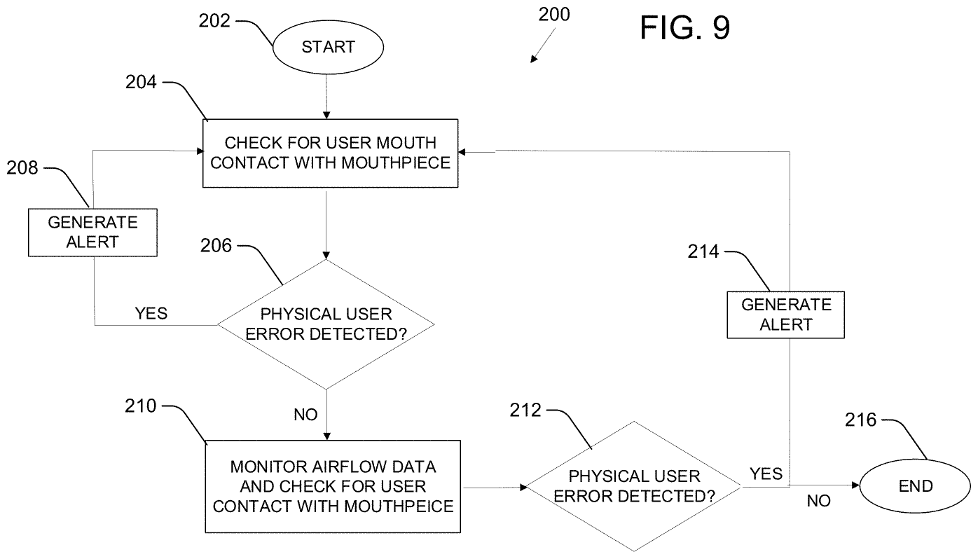

FIG. 9 illustrates a process 200 for mitigating physical user-errors during use of a spirometry system. FIG. 9 is described with regard to the spirometry system 100 shown in and discussed with regard to FIGS. 7A-8 above. The process 200 can start at step 202. At step 202, the spirometry system 100 can be communicatively coupled to the external device 103, such as via one or more user inputs to the user interface 130 of the spirometry system 100 or the user interface 150 of the external device 103 to initiate wireless communication therebetween. For example, a user can depress the first button 132 (FIG. 7A) to begin wireless pairing with the external device 103. In some examples, a user can communicatively couple the spirometry system 100 and the external device 103 using a direct (e.g., wired) connection, such as with a cable extending between the connector interface 144 and the connector interface 156.

Step 202 can also include generating an alert. For example, the controller 140 (FIG. 8) can interact with the control circuit 154 (FIG. 8) of the external device 103 (FIG. 8) to trigger the user interface 150 (FIG. 8) of the external device 103 to generable audible or visual instructions indicating that the user make contact with the proximal portion 104 (FIGS. 7A-7B) of the mouthpiece 102 (FIGS. 7A-7B). In an example, a user can depress the first button 132 (FIG. 7A), or otherwise interact with the user interface 130, to initiate a countdown timer indicating an amount of time remaining until the step 204 begins. Such a timer can be for example, but not limited to, about 1-3 seconds, 4-6 seconds, or 6-10 seconds.

The process 200 can include step 204. At step 204, the controller 140 can check for user mouth contact, such as by processing the at least one mouth contact signal received from the one or more contact sensors 124 (FIG. 7A & 8). For example, the controller 140 can compare the at least one mouth contact signal to at least one criterion, such as a specified threshold value, corresponding to at least one of an amount of user lip contact or an amount of user tongue contact, to thereby determine an indication of an amount of user mouth contact with the one or more contact sensors 124. The at least one decision criterion need not include using a specified threshold value, but can be established in any other suitable way, such as by using a trained statistical or machine learning model or an algorithmic application of one or more criteria. For example, a statistical or machine learning model can be trained using “ground truth” input from a human respiratory therapist, to which any of a number of different artificial intelligence or machine learning techniques can be applied (e.g., Support Vector Machines (SVM) as an illustrative example). In an example, the at least one mouth contact signal can include a lip contact signal. In such an example, the at least one criterion can be a threshold value, such as indicative of user lip contact with all, or a selected minimum number of, the one or more contact sensors 124. For example, each of the one or more contact sensors 124 can generate a binary signal corresponding to a contact, or a no-contact, indication.

In an example, the at least one mouth contact signal can include a tongue contact signal. In such an example, at least one of the one or more contact sensors 124 can be a force sensor configured to generate a mouth contact signal corresponding to the amount of force a user's tongue is exerting on the at least one force sensor. In such an example, the threshold value can be a force value associated with pressure that a user's tongue can apply to at least one of the one or more contact sensors 124, such as a force of between about, but not limited to, 3.2 N to 54 N.

The process 200 can include step 206. At step 206, the controller 140 can decide whether a physical user error was detected based on the determined indication of the amount of mouth contact determined at step 204. If the controller 140 determines that the amount of user mouth contact was insufficient, the process 200 can proceed to step 208. At step 208, the controller 140 can cause the user interface 130 or the vibrator 148 to generate an audible, visual, tactile, or haptic alert, or the controller 140 can interact with the control circuit 154 to trigger the user interface 150 to generate an audible, visual, tactile, or haptic alert, to inform a user that their lip or tongue contact with the one or more contact sensors 124 was insufficient.

For example, if the alert is a visual alert, the user interface 150 of the external device 103 can spell out various related messages, such as including, but not limited to “your lips came off during the exam”, “your tongue came off during the exam!”, cough detected during the exam!”, “variable effort detected during the exam!”, “you started too late”, or “exam cancelled”. If the alert is an audible alert, the user interface 130 or 152 can emit a continuous or periodic audio signal at constant or changing frequency, pitch, volume, or tone, such as based on an amount of user contact with the one or more contact sensors 124. Similarly, if the alert is a tactile or haptic alert, the vibrator can activate and either remain continuously active, or can activate and deactivate intermittently, at any of various speeds or vibratory frequencies.

If the controller 140 determines that the amount of user mouth contact was sufficient, the process 200 can proceed to step 210. At step 210, a spirometry test can be facilitated by the spirometry system 100. For example, the controller 140 can be configured to interact with the interface 150 of the external device 103 to guide a user through any of various operations of the spirometry test. During the spirometry test, the controller 140 can continuously or intermittently check for user mouth contact with the proximal portion 104 of the mouthpiece 102, such as described with regard to step 204 above, and concurrently, the controller 140 can monitor the airflow data (e.g., an airflow signal) from the airflow sensor 125 to both record user performance data and also check for additional user physical errors (e.g., non-user contact based errors), such as a late start to a test, a sub-maximal inhalation during a test, sub-maximal blast during a test, early user termination during a test, variable effort during a test or a cough during a test (such as by utilizing Riemann-based calculations to obtain the first and second derivative of the airflow signal), cessation of airflow during a test, tongue thrusting during a test, among others.

As the controller 140 can continuously process both the airflow signal and the at least one mouth contact signal during step 210, the controller 140 can decide whether a physical user error was detected at any time during step 210. Step 208 can further include checking calibration of the airflow sensor 125 (FIG. 8) to help prevent positive or negative calibration errors. If any type of physical error is detected during step 210, the process 200 can proceed to step 214. Step 214 can be similar to step 204 described above, at least in that step 214 can include the controller 140 causing the user interface 130 or the vibrator 148 to generate an audible, visual, tactile, or haptic alert, or the controller 140 interacting with the control circuit 154 to trigger the user interface 150 to generate an audible, visual, tactile, or haptic alert, to inform a user that their lip or tongue contact with the one or more contact sensors 124 was insufficient, or that their performance during the spirometry test included additional physical errors.

If the controller 140 determines that the amount of user mouth contact was sufficient and no physical errors were detected from the airflow signal, the process 200 can proceed to step 216 without proceeding to step 214. At step 216, the controller 140 can calculate PEF, FEV1, and FVC, or other pulmonary performance metrics and facilitate the transmission and storage of the spirometry test data to the external device 103, such as to be displayed to a user on the user interface 150, or on a display screen of the user interface 130.

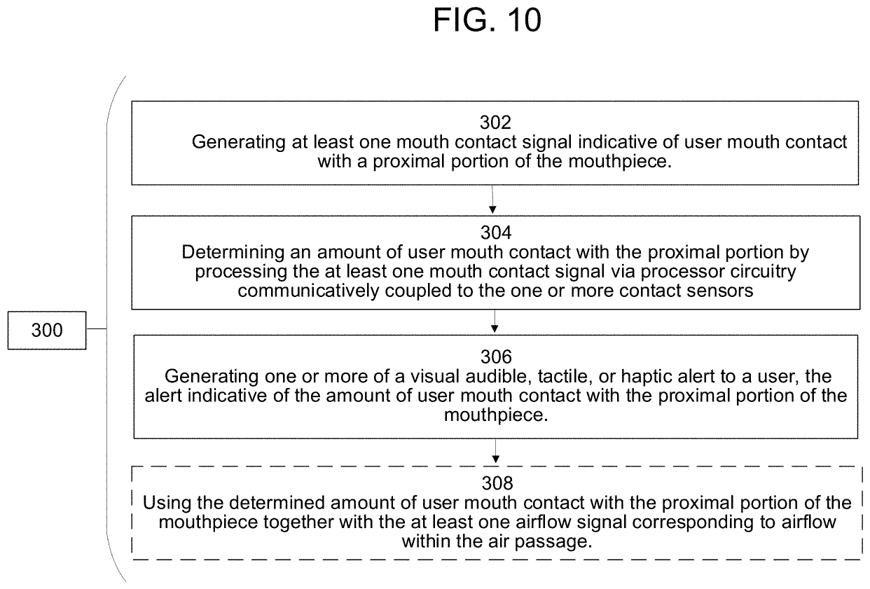

FIG. 10 illustrates a flow chart of an example method 300 for mitigating physical user-errors during use of a spirometry system. The steps or operations of the method 300 are illustrated in a particular order for convenience and clarity. The discussed operations can be performed in parallel or in a different sequence without materially impacting other operations. The method 300 as discussed includes operations that can be performed by multiple different actors, devices, and/or systems. It is understood that subsets of the operations discussed in the method 300 can be attributable to a single actor device, or system, and could be considered a separate standalone process or method.

The method 300 can begin with operation 302. Operation 302 can include generating at least one mouth contact signal indicative of user mouth contact with a proximal portion of the mouthpiece. For example, a user can bring the proximal portion of the mouthpiece into contact with their lips or tongue to contact one or more contact sensors distributed on the proximal portion of the mouthpiece to thereby generate an electrical signal.

Operation 302 can optionally include generating a plurality of lip contact signals using respective contact sensors comprising at least one of: a resistive contact sensor, an optical proximity sensor, a capacitive contact sensors. For example, the one or more contact sensors can include a plurality of individual contact sensors arranged about the proximal portion, each configured to generate an individual lip contact signal, such as a binary signal, when a user brings the mouthpiece into contact with their lips. The operation 302 can optionally include generating a plurality of lip contact signals individually generated using a corresponding contact sensor, and a tongue contact signal to provide an indication of at least one of an amount of user tongue contact or tongue force the proximal portion of the mouthpiece. For example, the one or more contact sensors can include a plurality of individual contact sensors arranged about the proximal portion and configured to each generate an individual lip contact signal, such as a binary signal, when a user brings the mouthpiece into contact with their lips, as well as a contact or force sensor positioned to generate a tongue contact signal, such as including a force value, when a user brings the mouthpiece into contact with their tongue.

The method 300 can include operation 306. Operation 306 can include generating one or more of a visual, audible, tactile, or haptic alert to a user, the alert indicative of the amount of user mouth contact with the proximal portion of the mouthpiece. For example, the spirometry system can be communicatively coupled to an external device, and a controller of the spirometry system can be configured to trigger a user interface of the external device to generate a visual, audible, tactile, or haptic alert to a user based on the amount of user mouth contact. In another example, the spirometry system can include a user interface, such as located on a handle or housing of the spirometry system, configured to generate a visual, audible, tactile, or haptic alert to a user based on the amount of user mouth contact. Such alerts could indicate, but are not limited to, insufficient or otherwise incomplete lip sealing with the proximal portion of the mouthpiece, or tongue thrusting or other undesirable tongue movement during a test.

The operation 306 can optionally include comparing the at least one mouth contact signal to at least one criterion to provide an alert. For example, the spirometry system can be configured to compare the at least one mouth contact signal to a threshold value, such as indicative of user lip contact with all, or a selected minimum number of, the one or more contact sensors, or can be a force value associated with pressure that a user's tongue can apply to least one of the one or more contact sensors.

The operation 306 can optionally include operation 308. Operation 308 can include using the determined amount of user mouth contact with the proximal portion of the mouthpiece together with the at least one airflow signal corresponding to airflow within the air passage. For example, the spirometry system can be configured to process both of the mouth contact signal and an airflow signal concurrently to continuously monitor an amount of user mouth contact with a proximal portion of the mouthpiece and pressure data to identify additional physical user errors such as sub maximum inhalation, a late start, a sub-maximal blast, a cough, such as in the first second of a test, early termination, variable effort, cessation of airflow, extra breaths, or positive or negative calibration.

In view of all the above, the spirometry system of the present disclosure can provide benefits for both patients and physicians. By reducing the need for in-person visits for accurate spirometry assessments, patients can save time and reduce healthcare costs, and patients with ambulatory constraints such as the elderly can more easily obtain access to pulmonary function testing. Further, more frequent monitoring can improve patient outcomes by allowing physicians to adjust treatment plans more effectively, as well as better identify specific triggers for respiratory dysfunctions in patients suffering from conditions such as asthma.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable media or machine-readable media encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure.

This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

### NOTES AND EXAMPLES

The following, non-limiting examples, detail certain aspects of the present subject matter to solve the challenges and provide the benefits discussed herein, among others.

Example 1 is a spirometry system operable to measure airflow through an air passage, wherein the spirometry system can comprise: a mouthpiece, defining the air passage, the mouthpiece including one or more contact sensors distributed about a proximal portion of the mouthpiece and configured to generate at least one mouth contact signal indicative of user mouth contact with the proximal portion of the mouthpiece; an airflow sensor, in fluid communication with the air passage, the airflow sensor configured to generate an airflow signal corresponding to airflow within the air passage; and processor circuitry, communicatively coupled to the one or more contact sensors and to the airflow sensor, the processor circuitry configured to determine an indication of an amount of user mouth contact for use by the processor circuitry with a measurement of airflow by the airflow sensor.

In Example 2, the subject matter of Example 1 can include, wherein the one or more contact sensors being configured to provide an indication of an amount of user lip or tongue contact.

In Example 3, the subject matter of Examples 1-2 can include, wherein the processor circuitry is configured to determine an indication of an amount of user mouth contact by comparing the at least one mouth contact signal from the one or more contact sensors to at least one criterion corresponding to at least one of an amount of user lip contact or an amount of user tongue contact.

In Example 4, the subject matter of Examples 1-3 can include, wherein the processor circuitry is configured to differentiate between user lip contact or user tongue contact based on the at least one mouth contact signal.

In Example 5, the subject matter of Examples 1-4 can include, wherein the one or more contact sensors protrude laterally outward beyond an outer surface of the mouthpiece carrying the one or more contact sensors.

In Example 6, the subject matter of Examples 1-5 includes, wherein the one or more contact sensors extend flush with or embedded within an outer surface of the mouthpiece.

In Example 7, the subject matter of Examples 1-6 includes, a housing, coupled to the mouthpiece and encompassing the airflow sensor, and wherein at least one of the processor circuitry or the airflow sensor includes a wireless communication interface associated therewith.

In Example 8, the subject matter of Example 7 can include, wherein the processing circuitry is configured to trigger generating an audible, visual, tactile, or haptic alert based at least in part on the indication of the amount of user mouth contact.

In Example 9, the subject matter of Examples 1-8 can include, a user interface included in or coupled to the housing to provide an alert or other user output.

In Example 10, the subject matter of Examples 1-9 can include, a vibrator, and wherein the processor circuitry is configured to activate the vibrator based at least in part on the at least one mouth contact.

In Example 11, the subject matter of Examples 1-10 can include, wherein the airflow passage defines a central axis and the one or more contact sensors are located in an distributed arrangement around the central axis and spaced apart from each other by between about 27 and 135 degrees. degrees.

Example 12 is a spirometry system operable to measure airflow through an air passage, wherein the spirometry system can comprise: a mouthpiece defining the air passage and including a proximal portion, the proximal portion including a movable member configured to contact a tongue of a user and movably coupled to the proximal portion, the movable member translatable between a first position and a second position that are spaced apart from each other laterally with respect to a central axis defined by the air passage; a position sensor configured to generate at least one member position signal indicative of a position of the movable member; an airflow sensor, in fluid communication with the air passage, the airflow sensor configured to generate an airflow signal corresponding to airflow within the air passage; and processor circuitry, communicatively coupled to the position sensor and the airflow sensor, the processor circuitry configured to determine the position of the movable member for use with a measurement of airflow by the processor circuitry.

In Example 13, the subject matter of Example 12 can include, wherein a surface of the movable member forms or aligns with a portion of an outer surface of the mouthpiece defining the air passage when the movable member is in the second position.

In Example 14, the subject matter of Examples 12-13 can include, wherein the position sensor further comprises a tongue contact sensor including at least one of a capacitive contact sensor, resistive contact sensor, optical proximity sensor, resistive force sensor, or load cell.

In Example 15, the subject matter of Examples 12-14 can include, one or more contact sensors distributed about the proximal portion of the mouthpiece and configured to generate at least one mouth contact signal indicative of user mouth contact with the proximal portion of the mouthpiece, and wherein the processor circuitry is communicatively coupled to the one or more contact sensors and is configured to determine an indication of an amount of user mouth contact for use with a measurement of airflow by the airflow sensor.

Example 16 is a method for mitigating a physical user-error during use of a spirometry system operable to measure airflow through an air passage defined by a mouthpiece, wherein the method can comprise: generating at least one mouth contact signal indicative of an amount of user mouth contact with a proximal portion of the mouthpiece by one or more contact sensors; determining an amount of user mouth contact with the proximal portion by processing the at least one mouth contact signal via processor circuitry communicatively coupled to the one or more contact sensors; and generating one or more of a visual, audible, tactile, or haptic alert to a user, the alert indicative of the amount of user mouth contact with the proximal portion of the mouthpiece.

In Example 17, the subject matter of Example 16 can include, wherein generating the at least one mouth contact signal indicating an amount of mouth contact includes generating a plurality of lip contact signals from respective contact sensors comprising at least one of: a resistive contact sensor, an optical proximity sensor, a capacitive contact sensors.

In Example 18, the subject matter of Examples 16-17 can include, wherein generating the at least one mouth contact signal to provide an indication of an amount of user lip contact with a proximal portion of the mouthpiece includes generating a plurality of lip contact signals individually generated using a corresponding contact sensor and a tongue contact signal to provide an indication of at least one of an amount of user tongue contact or tongue force the proximal portion of the mouthpiece.

In Example 19, the subject matter of Examples 16-18 can include, wherein determining the amount of user mouth contact includes comparing the at least one mouth contact signal to at least one criterion to provide an alert.

In Example 20, the subject matter of Examples 16-19 can include, using the determined the amount of user mouth contact with the proximal portion of the mouthpiece together with the at least one airflow signal corresponding to airflow within the air passage.

Example 21 is at least one machine-readable medium including instructions that, when executed by processing circuitry, cause the processing circuitry to perform operations to implement of any of Examples 1-20.

Example 22 is an apparatus comprising means to implement of any of Examples 1-20.

Example 23 is a system to implement of any of Examples 1-20.

Example 24 is a method to implement of any of Examples 1-20.

What is claimed is:

1. A spirometry system operable to measure airflow through an air passage, the spirometry system comprising:
a mouthpiece, defining the air passage, the mouthpiece including one or more contact sensors distributed about a proximal portion of the mouthpiece and configured to generate at least one mouth contact signal indicative of user mouth contact with the proximal portion of the mouthpiece;
an airflow sensor, in fluid communication with the air passage, the airflow sensor configured to generate an airflow signal corresponding to airflow within the air passage; and
processor circuitry, communicatively coupled to the one or more contact sensors and to the airflow sensor, the processor circuitry configured to determine an indication of an amount of user mouth contact for use by the processor circuitry with a measurement of airflow by the airflow sensor.

2. The system of claim 1, wherein the one or more contact sensors is configured to provide an indication of an amount of user lip or tongue contact.

3. The system of claim 1, wherein the processor circuitry is configured to determine an indication of an amount of user mouth contact by comparing the at least one mouth contact signal from the one or more contact sensors to at least one criterion corresponding to at least one of an amount of user lip contact or an amount of user tongue contact.

4. The system of claim 1, wherein the processor circuitry is configured to differentiate between user lip contact or user tongue contact based on the at least one mouth contact signal.

5. The system of claim 1, wherein the one or more contact sensors protrude laterally outward beyond an outer surface of the mouthpiece carrying the one or more contact sensors.

6. The system of claim 1, wherein the one or more contact sensors extend flush with or embedded within an outer surface of the mouthpiece.

7. The system of claim 1, further comprising a housing, coupled to the mouthpiece and encompassing the airflow sensor, and wherein at least one of the processor circuitry or the airflow sensor includes a wireless communication interface associated therewith.

8. The system of claim 7, wherein the processor circuitry is configured to trigger generating an audible, visual, tactile, or haptic alert based at least in part on the indication of the amount of user mouth contact.

9. The system of claim 1, further comprising:
a housing, coupled to the mouthpiece and encompassing the airflow sensor; and
a user interface included in or coupled to the housing to provide an alert or other user output.

10. The system of claim 1, further comprising a vibrator, and wherein the processor circuitry is configured to activate the vibrator based at least in part on the at least one mouth contact.

11. The system of claim 1, wherein the airflow passage defines a central axis and the one or more contact sensors are located in an distributed arrangement around the central axis and spaced apart from each other by between about 27 and 135 degrees.

12. A spirometry system operable to measure airflow through an air passage, the spirometry system comprising:
a mouthpiece defining the air passage and including a proximal portion, the proximal portion including a movable member, configured to contact a tongue of a user and movably coupled to the proximal portion, the movable member being translatable between a first position and a second position that are spaced apart from each other laterally with respect to a central axis defined by the air passage;
a position sensor configured to generate at least one member position signal indicative of a position of the movable member;
an airflow sensor, in fluid communication with the air passage, the airflow sensor configured to generate an airflow signal corresponding to airflow within the air passage; and
processor circuitry, communicatively coupled to the position sensor and the airflow sensor, the processor circuitry configured to determine the position of the movable member for use with a measurement of airflow by the processor circuitry.

13. The system of claim 12, wherein a surface of the movable member forms or aligns with a portion of an outer surface of the mouthpiece defining the air passage when the movable member is in the second position.

14. The system of claim 12, wherein the position sensor further comprises a tongue contact sensor including at least one of a capacitive contact sensor, resistive contact sensor, optical proximity sensor, resistive force sensor, or load cell.

15. The system of claim 12, further comprising one or more contact sensors distributed about the proximal portion of the mouthpiece and configured to generate at least one mouth contact signal indicative of user mouth contact with the proximal portion of the mouthpiece, and wherein the processor circuitry is communicatively coupled to the one or more contact sensors and is configured to determine an indication of an amount of user mouth contact for use with a measurement of airflow by the airflow sensor.

16. A method for mitigating a physical user-error during use of a spirometry system operable to measure airflow through an air passage defined by a mouthpiece, the method comprising:
generating at least one mouth contact signal indicative of an amount of user mouth contact with a proximal portion of the mouthpiece by one or more contact sensors;
determining an amount of user mouth contact with the proximal portion by processing the at least one mouth contact signal via processor circuitry communicatively coupled to the one or more contact sensors; and
generating one or more of a visual, audible, tactile, or haptic alert to a user, the alert indicative of the amount of user mouth contact with the proximal portion of the mouthpiece.

17. The method of claim 16, wherein generating the at least one mouth contact signal indicating an amount of mouth contact includes generating a plurality of lip contact signals from respective contact sensors comprising at least one of: a resistive contact sensor, an optical proximity sensor, a capacitive contact sensor.

18. The method of claim 16, wherein generating the at least one mouth contact signal to provide an indication of an amount of user lip contact with a proximal portion of the mouthpiece includes generating a plurality of lip contact signals individually generated using a corresponding contact sensor and a tongue contact signal to provide an indication of at least one of an amount of user tongue contact or tongue force the proximal portion of the mouthpiece.

19. The method of claim 16, wherein determining the amount of user mouth contact includes comparing the at least one mouth contact signal to at least one criterion to provide an alert.

20. The method of claim 16, comprising using the determined amount of user mouth contact with the proximal portion of the mouthpiece together with the at least one airflow signal corresponding to airflow within the air passage.
